(19) United States
(12) Patent Application Publication
(10) Pub. No.: US 2005/0112744 A1
(43) Pub. Date: May 26, 2005

(54) Glucose dehydrogenase fusion proteins and their use in expression systems

(76) Inventors: Winfried Linxweiler, Gross-Umstadt (DE); Christa Burger, Darmstadtz (DE); Oliver Poeschke, Wiesbaden (DE); Uwe Hofmann, Seehim-Jujenheim (DE); Andrea Wolf, Mannheim (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/681,207

(22) Filed: Oct. 9, 2003

Related U.S. Application Data
(62) Division of application No. 09/913,494, filed on Aug. 16, 2001, now abandoned.

(30) Foreign Application Priority Data
Feb. 19, 1999 (DE) DE 199 06 920.4

(51) Int. Cl. C12N 9/02; C07H 21/04

(52) U.S. Cl. 435/189; 435/69.1; 435/320.1; 435/325; 536/23.2

(57) ABSTRACT

The invention relates to novel recombinant fusion proteins containing a protein sequence having the biological activity of glucose dehydrogenase as one of its constituents and to their utilization for simple and efficient detection of any type of proteins/polypeptides in SDS-Page gels and for quick optimization of expression systems that can express the above-mentioned proteins/polypeptides.

The invention relates to novel recombinant fusion proteins which comprise as one constituent a protein sequence having the biological activity of glucose dehydrogenase (GlcDH), and to their use for the simple and efficient detection of any proteins/polypeptides, which preferably serve as fusion partners, and for the rapid optimization of expression systems which are able to express the said proteins/polypeptides.

In this regard, GlcDH or the sequence having the biological activity of GlcDH assumes the role of a marker or detector protein. A particular characteristic of this enzyme is exceptional stability to denaturing agents such as SDS. GlcDH as marker or detector protein shows undiminished enzymatic activity even after the reducing and denaturing conditions of SDS-PAGE gels. Fusion proteins comprising GlcDH can therefore be detected using a sensitive enzymatic reaction based on this surprising behaviour. It is thus also possible with GlcDH as marker for the required expressed protein to be detected rapidly, at low cost and efficiently.

It is furthermore possible in a number of cases for (GlcDH-protein/polypeptide fusion proteins to be expressed in higher yield and stability, especially in E. coli, than without GlcDH. Corresponding fusion proteins can thus be used per se for obtaining and preparing proteins/polypeptides.

The in vivo expression of recombinant proteins is playing an ever increasing part in biotechnology. The ability to obtain, purify and detect cloned gene products from pro- and eukaryotic expression systems such as, for example, bacterial, yeast, insect or mammalian cells is frequently also used for studies of protein structure and function, of protein-protein and protein-DNA interactions, and antibody production and mutagenesis. It is possible with the aid of the DNA recombination technique to modify natural proteins specifically to improve or alter their function. The recombinant proteins are synthesized in expression systems which are continually being further developed and which can be optimized at many different points in the system.

The overall process of recombinant protein synthesis can be divided into two sections. In a first step there is molecular biological isolation of the gene and expression of the target protein, and in the next step there is detection and purification from the recombinant cells or their growth medium. At the molecular level, the gene of a protein is cloned into an expression vector provided for this purpose and then inserted into a host cell (pro- or eukaryotic cell) and expressed therein. Bacterial cells prove in this connection to be simple and cost-effective systems affording high yields. The host cell most frequently employed is the Gram-negative bacterium E. coli.

The aim of expression of foreign genes in E. coli is to obtain the largest possible amount of bioactive recombinant proteins, which is called overexpression. It is known that eukaryotic foreign proteins may lose their biological activity during this through aggregation, as inclusion bodies, through incorrect folding or proteolytic degradation. One possibility of avoiding these frequently occurring difficulties is for the expressed proteins to be expelled from the cell as secreted proteins or else for so-called fusion proteins to be used, through which insoluble recombinant proteins may be present in soluble form in the cell.

In order to investigate the function of proteins and their interaction partners which are important for the function, proteins are usually expressed in eukaryotic cells. The post-transcriptional modifications which are important for the function, and the correct compartmentation can take place therein. In addition, other proteins important for the correct folding and processing are present.

Eukaryotic expression systems are also appropriate for expressing relatively large proteins and proteins which require post-transcriptional modifications such as, for example, S—S bridge formation, glycosylation, phosphorylation etc. for correct folding. Since these systems are usually complicated and costly, and the expression rate is below that of E. coli, it is particularly important to have a detection system which is rapid, reliable, sensitive and reasonably priced.

Numerous gene fusion systems exist for detecting foreign proteins which have been formed by recombination and whose biological function is unknown. In these, the expressed fusion protein is detected via the fusion protein portion whose function is known.

A sensitive detection system is necessary in order to determine correct expression, the amount expressed, the molecular weight and the functional activity of the fusion protein formed. The number of proteins of unknown function is increasing rapidly and it is becoming increasingly important to develop rapid and cost-effective detection systems therefor. With most gene fusion systems, immunological methods such as, for example, the enzym-linked immunosorbent assay (ELISA) or the Western blot are employed, in which fusion proteins formed by recombination are detected with the aid of specific antibodies.

However, corresponding fusion proteins not only have the described advantage that the foreign protein can easily be detected and analysed indirectly; on the contrary in many cases they allow the required protein to be expressed in higher yields than would be the case without its fusion partner. Each fusion partner has advantages, which it is not uncommonly able to transfer to the other partner, in a particular expression system. Thus, for example, the sensitivity of some proteins to protolytic [sic] degradation can be reduced when it is [sic] in the form of a fusion protein. Fusion proteins also frequently have more favourable solubility and secretion properties than the individual components.

There are thus numerous reasons for carrying out gene fusions for expressing recombinant proteins in heterologous hosts. These are: increasing the solubility of foreign proteins, increasing the stability of soluble foreign proteins, localizing the foreign protein in a specific section of the cell, rapid isolation of foreign proteins by simplified purification strategies, possibility of the fusion protein to be specifically cleaved off, possibility of rapid detection of the foreign protein from unpurifed cell extracts.

At present there are many functional tests for testing the expression of recombinant proteins with the aid of gene fusion systems. These comprise simple tests which usually make direct detection possible from unpurified cell extracts. However, the test systems differ considerably in the time taken, throughput and sensitivity.

For the abovementioned purposes it is possible to distinguish two types of fusion proteins. On the one hand fusion proteins which consist of the required protein and a usually short oligopeptide. This oligopeptide (“tag”) functions as a marker or recognition sequence for the required protein. A tag may additionally simplify purification.

The main use of the tag is firstly in the testing of expression and secondly in protein purification. One example thereof is the so-called His tag which consists of a peptide sequence which has six consecutive histidine residues and is directly linked to the recombinant protein. With the aid of the attached His residue it is easily possible to purify the fusion protein on a metal affinity column (Smith et al., 1988). This His tag is detected simply with the aid of the highly specific monoclonal antibody His-1 (Pogge v. Strandmann et al., 1995). Another marker used in fusion proteins is GFP, a green fluorescent protein (GFP) which is derived from the jellyfish Aequorea victoria and is employed as bioluminescent protein in various biotechnological applications (Kendall and Badminton, 1998; Chalfie et al., 1994; Inouye et al., 1994). It can easily be detected by its autofluorescence in living cells, gels and even live animals.

Further examples of tags, which will not be explained further, are the Strep-tag system (Uhlen et al., 1990) or the myc epitope tag (Pitzurra et al., 1990).

The main use of fusion proteins consisting of a recombinant protein and a functionally active protein is, besides the detection described above, in the simplified purification of the expressed fusion proteins. Among these, various systems are known, some of which will be mentioned briefly hereinafter.

In the GST system, fusion vectors make it possible to express complete genes or gene fragments in a fusion with glutathione S-transferase. The GST fusion protein can easily be purified from the cell lysates by affinity chromatography on glutathione-Sepharose (Smith, Johnson, 1988). A biochemical and an immunological detection is available. The maltose-binding protein in the MBP system is a periplasmic protein from E. coli which is involved in transporting maltose and maltodextrins through the bacterial membrane (Kellermann et al., 1982). It has been used in particular for expressing and purifying alkaline phosphatase on a crosslinked amylose column. The intein system is specifically suitable for rapid purification of a target protein. The intein gene has the sequence for the intein chitin binding domain (CBD), through which the fusion protein can be bound directly from the cell extract onto a chitin column and thus purified (Chong et al., 1997).

Glucose dehydrogenase (GlcDH) is a key enzyme during the early phase of sporulation in Bacillus megaterium (Jany et al., 1984). It specifically catalyses the oxidation of β-D-glucose to D-gluconolactone, with NAD⁺ or NADP⁺ acting as coenzyme. Apart from bacterial spores, the enzyme also occurs in the mammalian liver Two mutually independent glucose dehydrogenase genes (gdh) exist in B. megaterium M1286 (Heilmann et al., 1988). GdhA and gdhB differ considerably in nucleotide sequence, whereas GlcDH-A and GlcDH-B have, despite differences in the protein sequence, approximately the same substrate specificity. Further information and the corresponding DNA and amino acid sequences are also to be found, for example, in EP-B 0290 768.

The systems described above for detecting foreign proteins which have been formed by recombination and whose biological function is either unknown or inadequately known are usually complicated and time-consuming. This means that improvement and optimization of the expression conditions often cannot be done quickly or simply enough.

It is therefore a great advance to have developed a fusion protein partner which makes faster detection of the fusion protein possible, and does not have the disadvantages described in the state of the art for comparable systems.

It has now been found that fusion proteins which comprise GlcDH or a sequence which [lacuna] the biological activity of GlcDH are outstandingly suitable for detecting any required “foreign or target protein” more quickly, simply and thus efficiently than using the state of the art described. This property is based on the surprising finding that GlcDH retains its enzymatic activity under conditions under which other enzymes are inactivated (for example with SDS-PAGE).

The possibility of purifying dehydrogenases on immobilized dyes such as Cibachron Blue 3 G or other NAD-analogous compounds such as aminohexyl-AMP, which are similar, owing to their structure, to the NAD⁺coenzyme and likewise bind to all dehydrogenases, is known.

Thus, as part of a fusion protein, glucose dehydrogenase facilitates, owing to its affinity for the dyes which are, for example, immobilized on a gel and which are commercially available, the purification of the fusion protein in one step. It is furthermore possible to detect GlcDH as constituent of a fusion protein by coupling the enzymatic reaction to a sensitive colour reaction, preferably with iodophenyl-nitrophenyl-phenyltetrazolium salt (INT) or nitro blue tetrazolium salt (NBT) (under the stated conditions), which further simplifies indirect detection of the foreign protein. The method for staining GlcDH as marker enzyme additionally has the advantage that it does not impede the customary staining of proteins using, for example, Coomassie dyes or silver staining in the same gel.

In one embodiment of the present invention, the fusion protein consists of, besides GlcDH and the foreign protein, also a tag peptide which can be used for additional characterization of the proteins bound to the tag peptide. The characterization takes place, for example, via the polyhistidine tag, which is recognized as antigen by specific antibodies. Detection of the resulting antigen-antibody complex then takes place, for example, using a peroxidase (POD)-labelled antibody via methods known per se. The bound peroxidase produces, after addition of an appropriate substrate (for example ECL system, Western Exposure Chemiluminescent Detection System, from Amersham), a chemiluminescent product which can be detected using a film suitable for this purpose. The immunological detection can, however, also take place by a technique known per se, through a specific antibody tag, for example the myc tag. The polyhistidine tag, alone or in combination with the myc tag, additionally has the advantage that the fusion protein can be purified by binding to a metal chelate column.

However, the GlcDH fusion protein can also be purified and isolated by affinity chromatography directly on a specific anti-GlcDH antibody which has, for example, been immobilized on a chromatography gel such as agarose.

Another advantage of the invention is that GlcDH can be expressed in soluble form in high yields, preferably in E. coli by the known expression systems (see above). Thus, recombinant glucose dehydrogenase from Bacillus megaterium M1286 has been successfully expressed with high enzymatic activity in E. coli (Heilmann 1988). The expression of other eukaryotic genes in E. coli is often limited by the instability of the polypeptide chain in the bacterial host. Incorrect folding may lead to aggregation (“inclusion bodies”), reduced or absent biological activity and proteolytic degradation A corresponding fusion gene in which the GlcDH gene or a fragment having the biological activity of GlcDH has been ligated to the gene for the required foreign protein, can now be converted according to the invention into the fusion protein with virtually unchanged expression rate and yield compared with the GlcDH gene without fusion partner. This can also take place when expression of the foreign protein on its own is not possible per se or is possible only in reduced yields or only in an incorrectly folded state or only by use of additional techniques. It is thus possible to obtain the required foreign protein by subsequent elimination of the marker protein GlcDH or of the target protein, for example with endoproteases.

An example according to the invention of a target protein which can be expressed successfully as fusion protein together with GlcDH in E. coli is tridegin. Tridegin is an extremely effective peptide inhibitor for blood coagulation factor XIIIa and is derived from the leech Haementeria ghilianii (66 AA, 7.6 kD; Finney et al., 1997).

However, there are no restrictions to be mentioned according to the invention in relation to the nature and the properties of the foreign protein employed.

The invention is not restricted just to the expression of the fusion proteins according to the invention in E. coli. On the contrary, such proteins can also be synthesized advantageously using methods known per se and appropriate stable vector constructs (for example with the aid of the human cytomegalovirus (CMV) promoter) in mammalian, yeast or insect cells with good expression rates.

It is accordingly possible from the above description to characterize the invention in summary as follows and as indicated in the claims:

The invention thus relates to a recombinant fusion protein consisting of at least a first and second amino acid sequence, the first sequence having the biological activity of glucose dehydrogenase. The invention particularly relates to a corresponding recombinant fusion protein in which the said second sequence is any recombinant protein/polypeptide X or represents parts thereof.

The fusion proteins according to the invention may additionally comprise recognition sequences, in particular tag sequences. The invention thus relates further to a corresponding fusion protein which may additionally have at least one other tag sequence or recognition sequence suitable for detection.

The fusion proteins according to the invention have a wide variety of possible uses. In this connection, glucose dehydrogenase with its properties plays the crucial part. Thus, the invention relates to the use of glucose dehydrogenase as detector protein for any recombinant protein/polypeptide X in one of the said fusion proteins. The invention further relates to the use of glucose dehydrogenase in a detection system for the expression of a recombinant protein/polypeptide X as constituent of a corresponding fusion protein. The invention further relates to the use of GlcDH for detecting protein-protein interactions, where one partner corresponds to the recombinant protein/polypeptide X as defined hereinbefore and hereinafter. Finally, GlcDH may serve according to the invention as detector protein for any third j protein/polypeptide which is not a constituent of the fusion protein but is able to bind to the second sequence of the protein/polypetide X in the said fusion protein. GlcDH can furthermore be employed as marker protein for a partner in ELISA systems, Western blot and related systems.

Since the invention employs recombinant techniques it also, of course, comprises corresponding vectors, host cells and expression systems. The invention relates not only to these vectors and host cells as such but also to the use of corresponding expression vectors in optimizing the expression of a recombinant protein/polypeptide X in a recombinant preparation process, and to the use of a corresponding host cell in optimizing the expression of a recombinant protein/polypeptide X in such a preparation process.

The invention also relates to a method for the rapid detection of any recombinant protein/polypeptide X by gel electrophoresis, in particular SDS-PAGE gel electrophoresis, where a corresponding fusion protein is prepared and fractionated by gel electrophoresis, and the recombinant protein/polypeptide to be detected is visualized in the gel via the enzymic activity of glucose dehydrogenase.

Employed according to the invention in this connection to detect the enzymic activity of glucose dehydrogenase is a colour reaction based on tetrazolium salts, in particular iodophenylnitrophenyl-phenyltetrazolium salt (INT) or nitro blue tetrazolium salt (NBT), it being possible for a general protein staining according to the state of the art to follow [sic] where appropriate before or after the said colour reaction has taken place.

The figures are briefly explained below

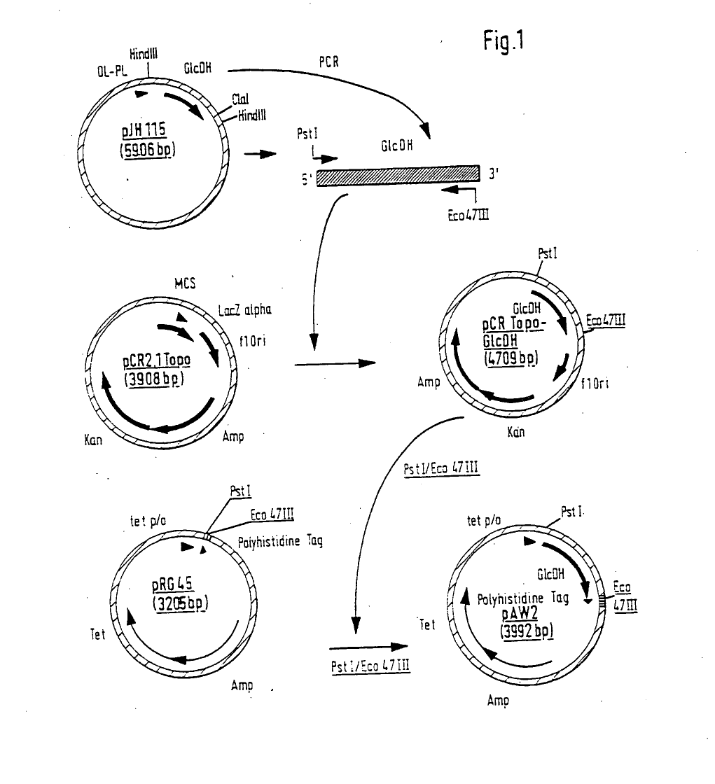

FIG. 1: Construction scheme for the vector pAW2. The vector contains the sequence for GlcDH. The complete sequence is depicted in Seq. Id. No. 1.

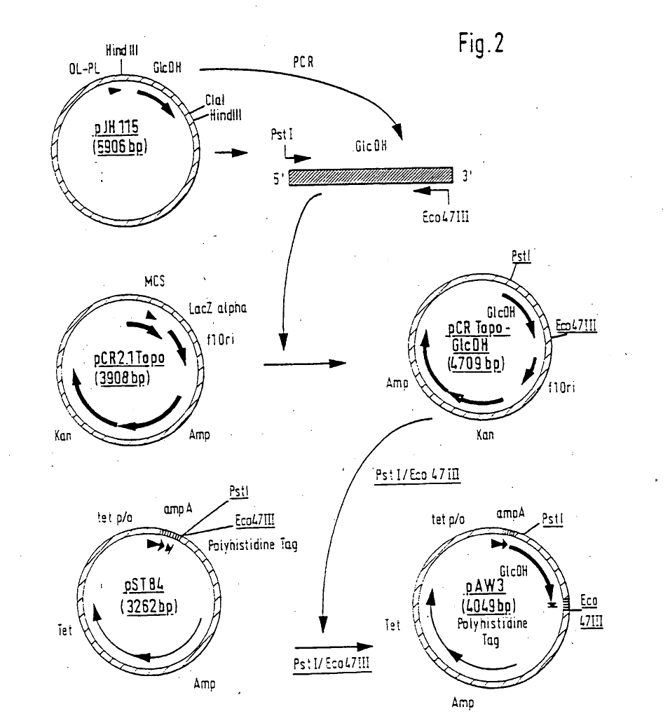

FIG. 2: Construction scheme for the vector pAW3.

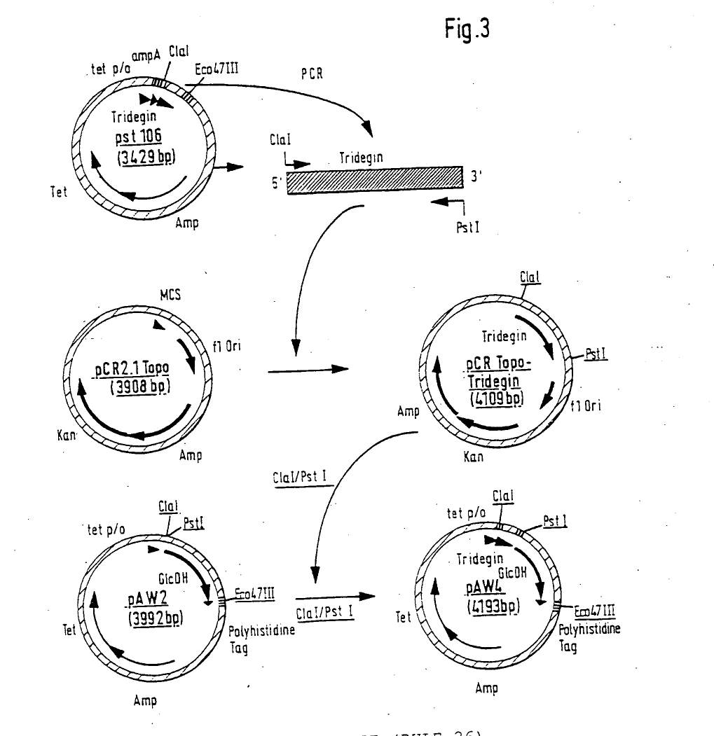

FIG. 3: Construction scheme for the vector pAW4. The vector contains the sequence for GlcDH and tridegin. The complete sequence is depicted in Seq. Id. No. 3.

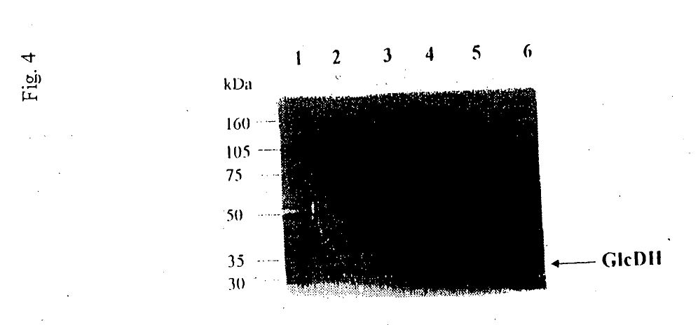

FIG. 4: Staining of GlcDH on an SDS-PAA gel. The staining method is described in detail in the examples. 1: Rainbow marker; 2: 0.1 μg of GlcDH; 3: 0.05 μg of GlcDH; 4: 0.001 μg of GlcDH; 5: lysate of HC11 cells; 6: prestained SDS marker.

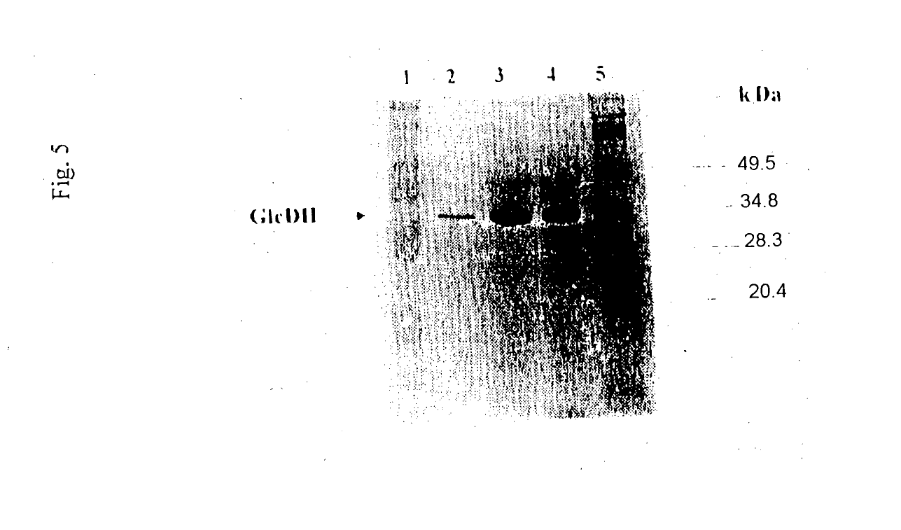

FIG. 5: Detection of the expressed GlcDH enzyme (15% SDS-PAA gel, INT stain); 1: Rainbow marker; 2: 0.2 μg of native GlcDH; 3: 10 μl of cell extract/1 ml of clone 2 suspension; 4: 10 μl of cell extract/1 ml of clone 1 suspension; 5: prestained SDS marker; cell extract volume: 100 μl.

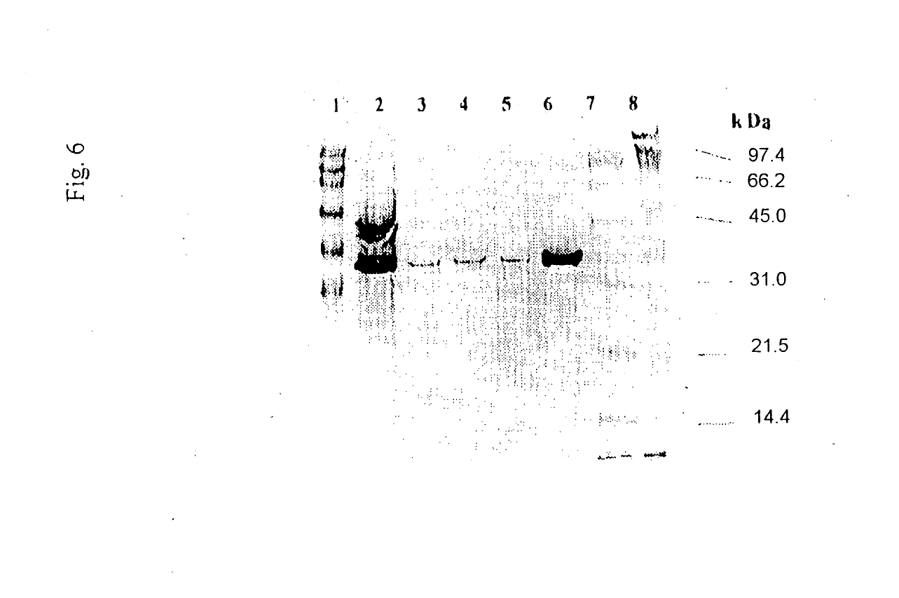

FIG. 6: Serial dilutions from pAW2 expression (15% SDS-PAA gel, INT stain); 1: Rainbow marker; 2: 10 μl of cell extract/100 μl of suspension; 3: 10 μl of cell extract/1:5 dilution; 4: 10 μl of cell extract/1:10 dilution; 5: 10 μl of cell extract/1:20 dilution; 6: 0.5 μg of GlcDH; 7: broad-range SDS marker; 8: prestained SDS marker; cell extract volume: 100 μl.

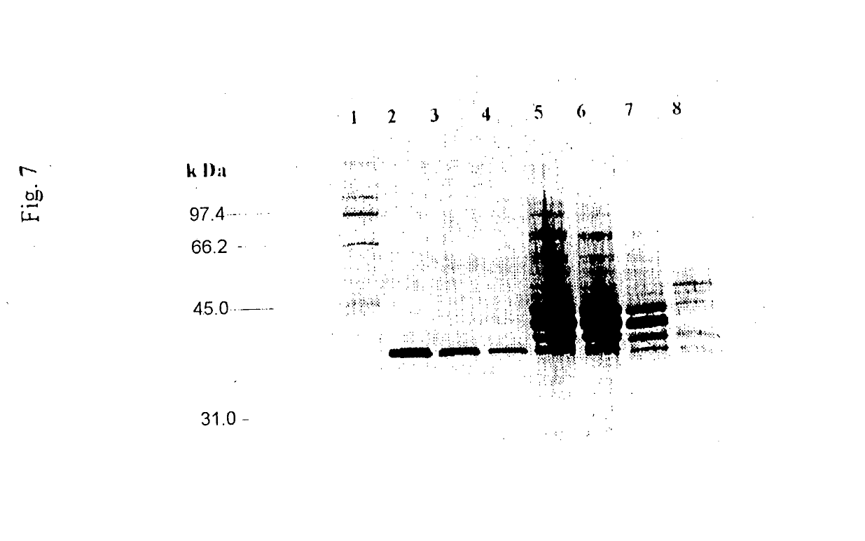

FIG. 7: Detection of the expressed tridegin/GlcDH fusion protein (10% SDS-PAA gel, INT/CBB); 1: broad-range SDS marker; 2: 1 μg of GlcDH; 3: 0.5 μg of GlcDH; 4: 0.1 μg of GlcDH; 5: 500 μl of cell extract; 6: 200 μl of cell extract; 7: 100 μl of cell extract; 8: 500 μl of cell extract (pAW2 expression); cell extract volume: 100 μl.

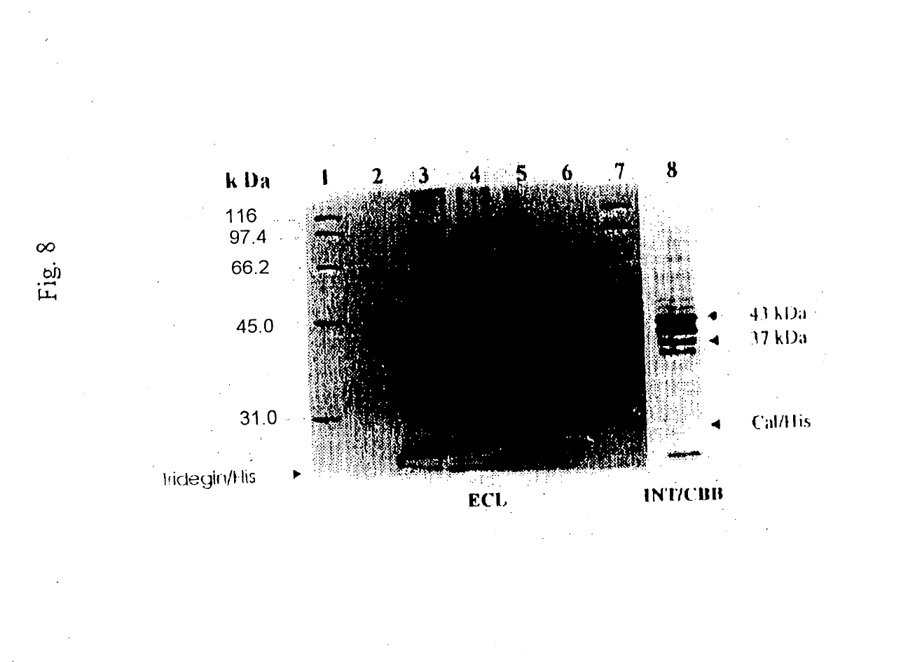

FIG. 8: Immunodetection of tridegin/His and tridegin/His/GlcDH fusion protein (from 10% SDS-PAA gel, ECL detection) and comparison with tridegin/His/GlcDH (10% SDS-PAA gel, INT-CBB stain); 1: broad-range marker; 2: 1 ml of cell extract (pAW2 expression); 3: 100 μl of cell extract (pST106 expression); 4: 200 μl of cell extract (pST106 expression); 5: 300 μl of cell extract (pAW4 expression); 6: 2.5 μg of calin-His positive control; 7: broad-range marker; 8: 100 μl [lacuna] (pAW4 expression); cell extract volume: 100 μl.

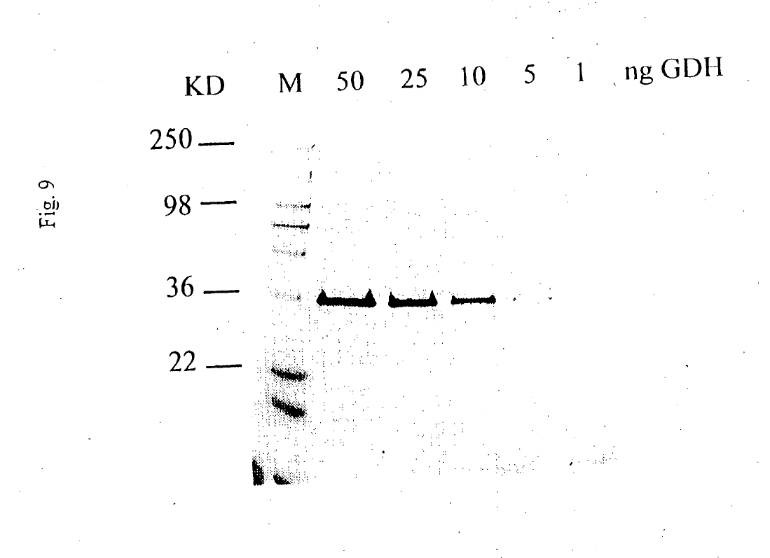

FIG. 9: SDS gel which explains the sensitivity of the detection of GlcDH. 1, 5, 10, 25 and 50 ng of GlcDH and molecular weight markers (left-hand column) are plotted.

The abbreviations used hereinbefore and hereinafter are explained below

| | |
|---|---|
| A | adenine |
| Aₓ | absorption at x nm |
| Ab | antibody |
| Amp | ampicillin |
| AP | alkaline phosphatase |
| APS | ammonium peroxodisulphate |
| AA | amino acid |
| bla | β-lactamase gene |
| BIS | N,N′-methylenebisacrylamide |
| bp | base pairs |
| BSA | bovine serum albumin |
| C | cytosine |
| cDNA | copy (complementary) DNA |
| CBB | Coomassie Brilliant Blue |
| CIP | calf intestinal phosphatase |
| dNTP | 2′-deoxyribonuceloside [sic] 5′-triphosphate |
| ddNTP | 2′,3′-deoxyribonuceloside [sic] 5′-triphosphate |
| DMF | dimethylformamide |
| DMSO | dimethyl sulfoxide |
| DNA | deoxyribonucleic acid |
| dsDNA | double-stranded DNA |
| DTT | dithiothreitol |
| ECL | Exposure ™ Chemiluminescence |
| EDTA | ethylenediamine-N,N,N′,N′-tetraacetic acid, |
| | disodium salt |
| ELISA | enzyme-linked immunosorbent assay |
| EtBr | ethidium bromide |
| EtOH | ethanol |
| f.c. | final concentration |
| FACS | fluorescent-activatet [sic] cell sorting |
| G | guanine |
| GFP | green fluorescent protein |
| GlcDH | glucose dehydrogenase (protein) |
| gdh | glucose dehydrogenase (gene) |
| GST | glutathione S-transferase |
| His | histidine residue |
| HRP | horseradish peroxidase |
| IB | inclusion body |
| IgG | immunoglobulin G |
| INT | iodonitrotetrazolium violet |
| kb | kilobase pairs |
| kD | kilodalton |
| mA | milliampere |
| m-RNA | messenger RNA |
| MBP | maltose-binding protein |
| MCS | multiple cloning site |
| Mᵣ | relative molecular weight |
| NAD(P) | nicotinamide adenine dinucleotide (phosphate), |
| | free acid |
| Odₓ | optical density at x nm |
| ompA | outer membrane protein A |
| ori | origin of replication |
| PAA | polyacrylamide |
| PAGE | polyacrylamide gel electrophoresis |
| PCR | polymerase chain reaction |
| POD | peroxidase |
| PVDF | polyvinylidene difluoride |
| RNA | ribonucleic acid |
| RNAse | ribonuclease |
| rpm | revolutions per minute |
| rRNA | ribosomal RNA |
| RT | room temperature |
| SDS | sodium dodecyl sulfate |
| ssDNA | single-stranded DNA |
| Strep | streptavidin |
| T | thymine |
| Tₘ | melting point (DNA duplex) |
| t-RNA | transfer RNA |
| Taq | Thermophilus [sic] aquaticus |
| TCA | trichloroacetic acid |
| TEMED | N,N,N′,N′-tetramethylethylenediamine |
| Tet | tetracycline |
| Tris | tris(hydroxymethyl)aminomethane |
| U | unit of enzymic activity |
| U | uracil |
| UV | ultraviolet radiation |
| ON | overnight |
| V | volt |
| VIS | visible |
| w/v | weight per volume |

### REFERENCES

Aoki et al. (1996), FEBS Letters 384, 193-197
Banauch et al. (1975), Z. Klin. Chem. Klin. Biochem. Vol. 13., 101-107
Bertram, & Gassen (1991) Gentechnische Methoden, Eine Sammlung von Arbeitsanleitungen für das molekularbiologische Labor. Gustav Fischer Verlag, Stuttgart, Jena, New York
Brewer & Sassenfeld (1985), Trends in Biotechnology 3, No. 5, 119-122
Brown, T. A. (1993) Gentechnologie für Einsteiger: Grundlagen, Methoden, Anwendungen. Spektrum Akademischer Verlag, Heidelberg; Berlin; Oxford
Casadaban et al. (1990), Methods in Enzymology 100, 293
Chalfie et al. (1994), Science 263, 802-805
Chong, S. et al. (1997), Gene 192, 271-281
Collins-Racie et al. (1995), Biotechnology 13, 982-987
Di Guan et al. (1988), Gene 67, 21-30
Ettinger et al. (1996), Proc. Natl. Acad. Sci. USA 93, 13102-13107
Finney et al. (1997), Biochem. J. 324, 797-805
Gazitt et al. (1992), Journal of Immunological Methods 148, 159-169
Ghosh et al. (1995), Analytical Biochemistry 225, 376-378
Goeddel et al. (1979), Proc. Natl. Acad. Sci. U.S.A. 76, 106-110
Hafner & Hoff (1984), Genetik. Neubearbeitung, Schrödel-Verlag, Hanover
Harlow & Lane (1988), A Laboratory Manual, Cold Spring Harbor
Harris & Angal (1990) Protein purification applications: a practical approach. Oxford University Press, Oxford; New York; Tokyo
Heilmann et al. (1988), Eur. J. Biochem. 174, 485-490
Hilt et al. (1991), Biochimica et Biophysica Acta 1076, 298-304
Ibelgaufts, H. (1990) Gentechnologie von A bis Z. Erweiterte Ausgabe, VCH-Verlag, Weinheim
Inouye et al. (1994), FEBS Letters 341, 277-280
Itakura et al. (1977). Science 198, 1056-1063
Jany et al. (1984), FEBS Letters 165, no. 1, 6-10
Kellermann & Ferenci (1982), Methods in Enzymology 90, 459-463
Laemmli (1970), Nature 227, 680-685
La Vallie & McCoy, (1995), Curr. Opin. Biotechnol. 6, 501-506
Makino et al. (1989), Journal of Biological Chemistry 264, No. 11, 6381-6385
Marston (1986), Biochem. J. 240, 1-12
Moks et al. (1987), Biochemistry 26, 5239-5244
Okorokov et al. (1995), Protein Expr. Purif., 6, 472-480
Pharmacia Biotech 1: From Cells to Sequences, A Guide to PCR analysis of nucleic acids
Pharmacia Biotech 2: The Recombinant Protein Handbook, Principles and Methods
Pitzurra et al. (1990), Journal of Immunological Methods 135, 71-75
Pogge v. Strandmann et al. (1995), Protein Engineering 8. No. 7, 733-735
Sambrook et al., (1989) Molecular Cloning, A Laboratory Manual 1, Second Edition, Cold Spring Harbor Laboratory Press, USA
Sanger et al. (1977), Proc. Natl. Acad. Sci. U.S.A. 74, 5463-5467
Schein (1989), Bio/Technology 7, 1141-1149
Scopes (1994) Protein purification; principles and practice, 3^{rd} ed., Springer-Verlag, New York; Berlin; Heidelberg
Smith & Johnson (1988), Gene 67, 31-40
Smith et al. (1988), Journal of Biological Chemistry 263, No. 15, 7211-7215
Uhlén & Moks (1990), Gene Fusions for Purpose of Expression: An Introduction [12]. Methods in Enzymology 185, 129-143
Uhlén et al. (1983), Gene 23, 369

Unless specified otherwise, the methods and techniques used for this invention correspond to methods and processes sufficiently well known and described in the relevant literature. In particular, the disclosure contents of the abovementioned publications and patent applications, especially by Sambrook et al. and Harlow & Lane, and EP-B-0290 768, are comprised in the invention. The plasmids and host cells used according to the invention are as a rule exemplary and can in principle be replaced by vector constructs which are modified or have a different structure, or other host cells as long as they still have the constituents stated to be essential to the invention. The preparation of such vector constructs, and the transfection of appropriate host cells and the expression and purification of the required proteins correspond to standard techniques which are substantially well known and may likewise be modified according to the invention within wide limits.

The invention is described further below. Further details are explained in the examples.

The Bacillus megaterium GlcDH structural gene was modified by PCR with the plasmid pJH115 (EP 0290 768) acting as template. The amplified fragment (0.8 kb), which had a PstI recognition sequence at one end and an Eco47III recogition sequence at the other, was digested with these enzymes and cloned into the cytoplasmic (pRG45) or periplasmic (pST84) E. coli expression vector (FIGS. 1, 2). The resulting plasmids, pAW2 and pAW3, now had a GlcDH gene which encodes a protein of about 30 kD (261 AA) and is located downstream of the strong Tet promoter. The cytoplasmic pAW2 expression vector has a size of about 4 kb. The periplasmic pAW3 secretion vector is slightly larger and differs from pAW2 only by an omp A signal sequence which is upstream of the multiple cloning site (MCS) and makes it possible for the recombinant protein to be secreted into the periplasm. Both vectors additionally have an MCS with 12 different restriction cleavage sites which make in-frame cloning with the following His tag possible. The polyhistidine (6His) tag makes it possible for the recombinant protein to be purified on a metal affinity column. The vector pAW4 finally comprises the tridegin gene and the GlcDH gene, which were connected together by an MCS, and the polyhistidine (6His) tag which is ligated downstream to the GlcDH gene. The individual constructs are depicted in FIGS. 1, 2 and 3. However, the chosen plasmid constructs are only by way of example and do not restrict the invention. They may be replaced by other suitable constructs containing the DNA sequences mentioned. The preparation of the vectors and the clones and expression of the proteins are specified further in the examples.

The sensitivity of the activity staining was carried out [sic] for native GlcDH in a reduced SDS gel. For this purpose, serial concentrations were prepared with native GlcDH (c=1 mg/ml; A=200 U/ml), and a negative control was prepared. SDS-PAGE and activity staining using INT resulted in the SDS gel depicted in FIG. 3. It was possible with the test employed to detect GlcDH down to a concentration of 50 ng. The negative control, which contains no GlcDH, shows no band, as expected.

The exact molecular weight of the native GlcDH can be determined using marker proteins and with the aid of a calibration plot. To do this, the relative migration distances of the marker proteins were determined and plotted against their respective logarithmic molecular weights.

A procedure for the expressions carried out was as depicted in the scheme (Tab. 1):

TABLE 1
Transformation of the GlcDH expression vectors into W3110 cells

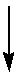

Preculture in LB(Amp) medium at 37° C. (12 h)

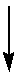

Cell growth at 37° C. in main culture with induction (5 h)

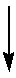

Centrifugation to obtain biomass

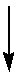

Suspension of the cells in 1x SDS loading buffer

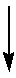

Cell disruption at 95° C. for 5 min

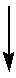

Cell extract can be used directly in SDS-PAGE (1 h)

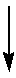

Activity staining of GlcDH in SDS gel (30 min)

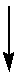

Gel band analysis

The plasmid pAW2/clone 9 (pAW2/K9) was transformed into the competent E. coli expression strain W3110, and two clones from the resulting transformation plate were used to inoculate a 5 ml preculture. Induction with anhydrotetracycline took place 2 h after inoculation of the main culture. Expression overall lasted 5 h and was stopped at an OD of 1.65 for clone 1 and 1.63 for clone 2. After SDS-PAGE and GlcDH activity staining, a strong GlcDH band (about 35 kD) was detectable for each clone from 1 ml of cell suspension.

No difference between the resulting GlcDH bands became evident when SDS-PAGE was carried out under reduced and non-reduced conditions. For this purpose, in each case 500 to 100 μl of the cell suspension were investigated in the SDS gel by GlcDH activity staining with INT.

In order to illustrate the sensitivity of the GlcDH activity staining compared with Coomassie staining, samples of 100 μl of cell suspension, and ⅕, 1/10 and 1/20 dilutions of the cell suspension were prepared. The final volume of the dilutions was likewise 100 μl. The resulting SDS gel was used, after the GlcDH activity staining, for a Coomassie staining to visualize further protein bands. The SDS gel resulting from this is depicted in FIG. 4. A distinct band is still evident at the 1/20 dilution using the GlcDH activity staining, whereas Coomassie-stained bands are now scarcely detectable.

The Haementeria ghilianii tridegin structural gene with coupled His tag was modified by PCR with the plasmid pST106 acting as template. The amplified fragment (0.25 kb), which is flanked by a ClaI recognition sequence and a PstI recognition sequence, was digested with these enzymes and cloned into the cytoplasmic E. coli GlcDH fusion vector pAW2. The resulting plasmid pAW4 now had a tridegin-His-GlcDH fusion protein gene which codes for a protein of about 44 kD and is located downstream of the strong Tet promoter. The cell extract from the E. coli strain W 3110 which comprises the cytoplasmic pAW4 plasmid was analysed by SDS-PAGE and GlcDH activity staining. It was possible therewith to detect several bands stained red-violet at 35, 37, 40 and 43 kD. The 43 kD band comprised the required tridegin-His-GlcDH fusion protein, although its molecular weight was somewhat less than the theoretical value of 44 kD. The remaining detectable bands were presumably produced by proteolytic degradation of the fusion protein in E. coli since the smallest stained and of 35 kD approximately corresponds to the size of GlcDH. It was possible on the basis of a size comparison to identify the 35 kD band which was formed as the His-GlcDH degradation product.

Carrying out [sic] expression kinetics revealed that proteolytic degradation of the formed fusion protein started 2 hours after induction of the Tet promoter with anhydrotetracycline, that is to say after this time additional bands were detectable in the SDS gel by activity staining. The formed fusion protein was not stable to E. coli proteases, which is shown by its relatively fast protein degradation. It was possible, by using the constructed periplasmic GlcDH fusion vector pAW3 to avoid proteolytic degradation of the fusion protein in the cell, because in this case the expressed fusion protein would be secreted into the periplasmic space between E. coli cells. E. Coli proteases are found mainly in the cytoplasm.

The sensitivity and specificity of the GlcDH fusion protein detection makes it possible for recombinant foreign proteins to be screened rapidly and simply. Sensitivity of the GlcDH detection system was determined using native GlcDH. Detection of native GlcDH activity resulted in a band stained red-violet at about 30-35 kD in the SDS-PAA gel.

Cytoplasmic expression in the E. coli strain W 3110 of the recombinant GlcDH from pAW2 showed the same molecular weight. Sensitivity comparison between native GlcDH and recombinant GlcDH was possible by comparing the band intensities.

The developed test system (see examples) additionally makes it possible to carry out double staining of the SDS gels. In the first staining there is specific detection of the GlcDH bands. The background staining can be followed by a conventional protein staining, for example a Coomassie staining of the remaining proteins. GlcDH surprisingly retains according to the invention under reducing conditions in the presence of SDS its complete activity, which makes rapid detection in the SDS gel possible.

It is furthermore possible according to the invention to increase the sensitivity of the detection of GlcDH activity by using nitro blue tetrazolium salt (NBT) as substrate for GlcDH. The reaction rate for the GlcDH detection using INT can, however, be increased further by using Triton X-100 (1% final solution) or adding NaCl (1 M final solution).

The recombinant fusion proteins tridegin/His and tridegin/His/GlcDH were obtained by expression of the pST106 and pAW4 plasmids (FIGS. 1, 2). After disruption of the cells in the relevant expression mixture, the samples were fractionated by SDS-PAGE and transferred to a membrane. The tridegin-His-GlcDH fusion protein was detectable immunologically via the His tag present therein by using an anti-^{RGS.}His antibody in a Western blot. The controls used were purified recombinant calin (leech protein) which has a terminal His tag, and the cell extract of the expressed recombinant GlcDH which has no His tag. The anti-^{RGS.}His antibody was able to detect a band at about 37 kD and another band at about 43 kD for the recombinant tridegin/His/GlcDH fusion protein (FIG. 6). Comparison of the sizes of the bands obtained with the bands obtained after activity staining in the SDS gel shows that the 43 kD band represents the tridegin-His-GlcDH fusion protein and the 37 kD band represents the His-GlcDH degradation product of the complete fusion protein. The calin/His tag protein produced a band at about 26 kD. The somewhat smaller recombinant tridegin/His tag protein produced a band at about 23 kD plus further bands indicating binding of the His antibody to other expressed proteins. The immunological detection with the anti-^{RGS.}His antibody thus proves that the protein detected at 43 kD and that detected at 37 kD contained a His tag. In addition, the size of the latter protein approximately corresponded to the theoretical size (36.5 kD) of the GlcDH protein with coupled His tag.

In addition to the detection of expression of the recombinant tridegin, the biological activity of tridegin as constituent of the tridegin-GlcDH fusion protein was investigated, in the specific case from pAW4. This test is based on the inhibition of factor XIIIa by native leech gland homogenate and purified tridegin (Finney et al., 1997). The modified test is described in the examples. As a control, the corresponding fusion protein from pST106 and the GlcDH protein from pAW2 were expressed. Comparison of the enzymic activity with recombinant tridegin expressed either as GlcDH-tridegin fusion protein or as tridegin-His tag in E. coli revealed negligible differences. In addition, the recombinant tridegin proteins from the two different expressions showed comparable biological activities to the native leech gland homogenate. It can be concluded from this that fusion with GlcDH has no interfering effect at all on the biological activity of the coexpressed foreign gene.

Tridegin itself (that is to say not as fusion protein) has no activity after E. coli expression and is formed as inclusion body: Expression of GlcDH in E. coli results in an enzyme with high specific activity and stability in soluble form. It was demonstrated in expression experiments that proteins which have a high solubility capacity on expression in E. coli increase the solubility capacity of foreign protein expression when they are fused to the latter (LaVallie, 1995). Fusion of tridegin to GlcDH in this case also increased the solubility of tridegin because it was possible by a biological detection in which tridegin inhibits factor XIIIa to detect the activity of tridegin after E. coli expression as tridegin-His-GlcDH fusion protein. The GlcDH fusion protein is expressed in high yield in E. coli.

The possibility of expressing cloned genes as fusion proteins containing a protein of known size and biological function markedly simplifies the detection of the gene product. For this reason, as mentioned in the introduction, numerous fusion expression systems have been developed with various detection strategies.

A comparison of the known systems with the GlcDH fusion system according to the invention in E. coli is shown in Tab. 2. In some systems, the N-terminal fusion protein can be cleaved off from the C-terminal target or foreign protein (Collins-Racie et al., 1995).

TABLE 2

| | MW | | |
|---|---|---|---|
| Tag/fusion partner | (kD) | Detection | Advantage |
| GlcDH | 30 | Function test | Rapid and low- |
| | | in the SDS | cost, direct |
| | | gel | detection in |
| | | | the SDS gel |
| His tag (Pogge v. | 1-7 | Western blot, | Small |
| Strandmann et al., | | ELISA | |
| 1995) | | | |
| Strep-tag (Uhlén et | 13 | Western blot, | Small |
| al., 1990) | | | |
| myc epitope | 1-2 | Western blot, | Small |
| (Pitzurra et al., | | ELISA | |
| 1990; Gazitt et | | | |
| al., 1992) | | | |
| IgG portions, Fc | 2-5 | Western blot, | Small, |
| (Moks et al., 1987; | | ELISA | selection of |
| Ettinger et al., | | | cells (FACS) |
| 1996) | | | |
| GFP (Chalfie et | 27 | Fluorescence, | Selection of |
| al., 1994; Inouye | | Western blot | cells even in |
| et al., 1994) | | | the culture |
| | | | dish, several |
| | | | detectable |
| | | | simultaneously |
| | | | (FACS) |
| Intein (Chong et | 48 | Western blott | Fusion partner |
| al., 1997) | | | can be deleted |
| GST (Smith, | 26 | Western blot, | Fusion partner |
| Johnson, 1988; Gosh | | colorimetric | can be deleted |
| et al., 1995) | | detection in | |
| | | solution | |
| MBP (Chu di Guan et | 40 | Western blot | Fusion partner |
| al., 1988; | | | can be deleted |
| Kellermann et al., | | | |
| 1982) | | | |

| | Pre- | Time | | | |
|---|---|---|---|---|---|
| Method | condition | taken | Throughput | Sensitivity | Information |
| GlcDH | GlcDH | about | moderate- | 50 ng | protein |
| detec- | function- | 3 h | high | | amount + |
| tion | ally | | | | protein |
| | active | | | | size |
| ELISA | 2 anti- | about | high | pg-ng | protein |
| | bodies | 1 day | | | amount |
| Western | 1-2 anti- | 1-2 | low | ng | protein |
| blot | bodies | days | | | size + |
| | Tag on | | | | protein |
| | the | | | | amount |
| | protein | | | | |

A very great advantage of the GlcDH detection system according to the invention is the fact that it does not require, such as, for example, for the Western blot detection, any antibodies or other materials such as, for example, membranes, blot apparatus, developer machine with films, microtitre plates, titre plate reader etc. This means that the detection of recombinant fusion proteins using the GlcDH system takes place very much more favourably and rapidly. It is possible with the aid of GlcDH detection to establish not only information about the amount of the expressed fusion protein but also the corresponding size of the fusion protein directly in the SDS-PAA gel without transfer to a membrane. If GlcDH activity is detectable in the fusion protein, the fusion partner ought also as a rule to be functionally active. GlcDH does not interfere with the folding of the fusion partner. The advantages of the GlcDH fusion protein system according to the invention are shown in a comparison hereinafter (Tab. 3 below) by selecting from the literature an efficient method for isolating and detecting a fusion protein obtained in E. coli.

The GlcDH fusion protein system according to the invention is furthermore particularly suitable for increasing the solubility of proteins which are formed, especially in E. coli, as inclusion bodys and therefore make subsequent protein purification difficult and costly. It is normally necessary to convert proteins formed as inclusion bodys into their native state by elaborate methods. This is unnecessary on use of the fusion proteins according to the invention.

In summary, the advantages of the fusion proteins according to the invention which are in use as GlcDH detection system are as follows.
Stability under SDS and reducing (denaturing) conditions
Sensitive GlcDH-specific enzymatic colour test
Sensitivity as far as at least 50 ng
Rapid detection directly in the SDS gel with determination of the molecular weight of the fusion partner
Possibility of additional protein stainings
Low-cost materials, little expenditure on apparatus
Good expression in E. coli, including that of the target protein with retention of the biological activity
Possibility of avoiding inclusion bodies of the foreign/target protein or other aggregates produced by incorrect folding

Possibility of purifying the fusion protein via affinity chromatography, for example on dyes (Cibacron Blue 3G)

TABLE 3

| | |
|---|---|
| Construction/transformation | Construction/transformation |
| of the protein A/GFP fusion | of the GlcDH/tridegin |
| vector | fusion vector |
| 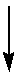 | 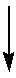 |
| Growth of the cells on LB | Preculture in LB(Amp) |
| agar plates at 37° C. | medium at 37° C. (12 h) |
| (1 day) | |
| 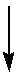 | 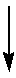 |
| Cell growth at 25° C. | Cell growth at 37° C. in main |
| (3 days) | culture with induction |
| | (5 h) |
| 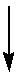 | 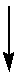 |
| Suspension of the cells In | Suspension of the cells in |
| buffer (pH 8.0) | SDS loading buffer |
| 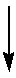 | 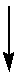 |
| Cell disruption and removal | SDS cell disruption at 95° C. |
| of cell detritus by | for 5 min |
| centrifugation | |
| 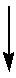 | 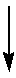 |
| SDS-PAGE for protein | SDS-PAGE (1 h) with cell |
| separation (1 h) | extract |
| 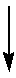 | 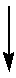 |
| Protein transfer to | |
| nitrocellulose membrane (1 h) | |
| 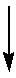 | |
| Blocking reaction (1 h) | GlcDH activity staining in |
| | the SDS gel (30 min) |
| 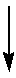 | 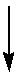 |
| Antibody reaction (1 h) | |
| 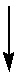 | |
| Incubation in protein A-GFP | |
| working buffer (20 min) | |
| 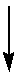 | |
| UV radiation | Analysis of the SDS gel |
| (365 nm)/analysis of the | with determination of the |
| blot | molecular weight |

The following examples illustrate the invention further without restricting it.

### EXAMPLE 1

| | | | |
|---|---|---|---|
| Primer | Sequence | Length | Use |

| | | | |
|---|---|---|---|
| GlcDH#1 | 5′-GCGCGAATTCATGTATACAGATTTANAAAGAT-3′ | 32 bases | PCR primer (attaches to the 5′ end of gdh |
| | | | and introduces an EcoRi cleavage site) |
| GlcDH#2 | 5′-GCGCTTCGAACTATTAGCCTCTTCCTGCTTG-3′ | 31 bases | PCR primer (attaches to the 3′ end of gdh |
| | | | and introduces an SfuI cleavage site) |
| GlcDH#3 | 5′-GCGCCTGCAGATGTATACAGATTTAAAAGAT-3′ | 31 bases | PCR primer (attaches to the 5′ end of gdh |
| | | | and introduces an PstI cleavage site) |
| GlcDH#4 | 5′-GCGCAGCGCTCTATTAGCCTCTTCCTGCTTG-3′ | 31 bases | PCR primer (attaches to the 3′ end of gdh |
| | | | and introduces an Eco47III cleavage site) |
| Tridegin#1 | 5′-GCGCATCGATATGAAACTATTGCCTTGCAAA-3′ | 31 bases | PCR primer (attaches to the 5′ end of |
| | | | tridegin and introduces a ClaI cleavage |
| | | | site) |
| Tridegin#2 | 5′-GCGCCTGCAGGTGATGGTGATGGTGATGCGA-3′ | 31 bases | PCR primer (attaches to the 3′ end of |
| | | | tridegin and introduces a PstI cleavage |
| | | | site) |
| pASK 75 UPN | 5′-CCATCGAATGGCCAGATCATTA-3′ | 22 bases | Sequencing primer (TRD 41 labelled at the 5′ |
| | | | end, attaches in tet p/o of pRG 45 and |
| | | | pST84) |
| PASK 75 RPN | 5′-TACCGGTAAACGGCAGACAAA-3′ | 21 bases | Sequencing primer (5′ IRD 41 labelled, |
| | | | attaches in lpp of pRG 45 and pST84) |
| T7 Seq.s | 5′-TAATACGACTCACTATAGGG-3′ | 20 bases | Sequencing primer (5′ IRD 41 labelled, |
| | | | attaches to the T7 priming site of |
| | | | pcDNA3.1/myc-His A, B, C |
| Rev Seq.as | 5′-TAGAAGGCACAGTCGAGG-3′ | 18 bases | Sequencing primer (5′ IRD 41 labelled, |
| | | | attaches to the BGH reverse priming site of |
| | | | pcDNA3.1/myc-His A, B, C) |

The above nucleotides were used according to the invention (Tab. 4).

Table 5 below summarizes the microorganisms used. All the microorganisms are derived from E. coli K12 and belong to risk group 1.

TABLE 5

| | Genus/ | | |
|---|---|---|---|
| Strain | species | Genotype | Literature |
| Top10F′ | E. coli | F′(lacI^{q}Tn10(Tet^{R}))mcrA | Top10F′ |
| One | | Δ(mrr-hsdRMS- | OneShot ™ Kit |
| Shot ™ | | mcrBC)Φ80lacZΔM15ΔlacX74 | from |
| Cells | | deoR recA1 araD139 | Invitrogen ® |
| | | Δ(ara-leu)7697 galU galK | |
| | | rpsL(StrR) endA1 nupG | |
| Epi- | E. coli | Δ(mcrA)183 Δ(mcrCB- | Stratagene's |
| curian | | hsdSMR-mrr) 173 endA1 | Competent |
| Coli ®XL1- | | supE44 thi-1 recA1 | Cells |
| Blue | | gyrA96 relA1 lac(F′ | |
| MRF′ | | proAB | |
| Cells | | lacI^{q}ZΔM15Tn10(Tet^{I})) | |
| TOP10 | E. coli | F⁻ mcrA Δ(mrr-hsdRMS- | TOPO TA |
| One- | | mcrBC) | Cloning ® Kit |
| Shot ™ | | Φ80lacZΔM15 ΔlacX74 | (Version C) |
| Cells | | recA1 deoR recA1 araD139 | from |
| | | Δ(ara-leu)7697 galU galK | Invitrogen ® |
| | | rpsL (Str^{R}) endA1 nupG | |
| W 3110 | E. coli | F⁻ λ⁻ WT E. coli | B. Bachmann, |
| | | | Bacteriol. |
| | | | Rev. 36(72) |
| | | | 525-557 |

Donor organism: M 7037 expression strain (E. coli N 4830/pJH 115) of 21.10.96 (supplied by Merck).
pJH 115: pUC derivative, 5.9 kb, O_{L}P_{L} promoter, gdh, to (terminator), galk (galactosidase gene), bla (β-lactamase gene), ori (origin of replication), 2 HindIII, 2 BamHI and one each EcoRI and ClaI cleavage site.

### EXAMPLE 2

Transformation of Plasmids Into Competent E. Coli Cells:

SOC medium: 20 g of Bacto tryptone, 5 g of Bacto yeast extract, 0.5 g of NaCl, 0.2 g of KCl ad 1 l ddH₂O, autoclave. Before use, add: 0.5 ml of 1 M MgCl₂/1 M MgSO₄ (sterile-filtered), 1 ml of 1 M glucose (sterile-filtered)

LB(Amp) agar plates: mix together 1 l of LB medium (without ampicillin) and 15 g of agar-agar, autoclave, cool to about 60° C. and 1 ml of ampicillin solution (100 mg/ml). Procedure:

| | | |
|---|---|---|
| Mixture | 1-5 μl | of ligation product or plasmid |
| | | DNA (5-50 ng/μl) |
| | 50 μl | of competent cells |
| | 450 μl | of SOC medium |

thaw competent cells on ice for 10 min
add DNA to the competent cells
incubate on ice for 30 min
heat shock: 30 sec at 42° C. (water bath)
place cells on ice for 2 min
add 450 μl of prewarmed SOC medium
incubate at 37° C. and 220 rpm for 1 h
streak 100 μl portions of the mixture onto a prewarmed LB(Amp) plate
incubate plates at 37° C. overnight

### EXAMPLE 3

TOPO-TA-Cloning® and Ligation

TOPO-TA-Cloning® is a five-minute cloning method for PCR products amplified with Taq polymerase.

The TOPO-TA-Cloning® kit (version C) supplied by Invitrogen was developed for direct cloning of PCR products. The system makes use of the property of thermostable polymerases which attach a single deoxyadenosine at the 3′ end of all duplex molecules in a PCR (3′-A overhang). It is possible with the aid of these 3′-A overhangs to link the PCR products directly to a vector which has 3′-T overhangs. The kit provides the pCR®2.1-TOPO vector which was specifically developed for this purpose. The vector is 3.9 kb in size and has a lacZ gene for blue/white selection, and ampicillin- and kanamycin-resistant genes. The cloning site is flanked on both sides by a single EcoRI cleavage site.

Ligation Mixture:

| | |
|---|---|
| 2 μl | of fresh PCR product (10 ng/μl) |
| 1 μl | of pCR ®-TOPO vector |
| 2 μl | of sterile water |
| 5 μl | total volume |

Carefully mix the mixture and incubate at RT for 5
Briefly centrifuge and place tube on ice
Employ ligation products immediately in the One Shot™ transformation

A 5 μl mixture without PCR product and consisting only of vector and water is used as control.

The One-Shot™ transformation was carried out by the following method:
Add 2 μl of 0.5 M β-mercaptoethanol to the 50 μl of One Shot™ TOP10 competent cells thawed on ice;
Add 2 μl of the TOPO-TA-Cloning® ligation per vial of competent cells;
Incubate on ice for 30 min
Heat shock: 30 sec at 42° C.;
Cool on ice for 2 min;
Add 250 μof SOC medium (RT);
Incubate the vials at 37° C. and 220 rpm for 30 min;
Streak 100 μl of each transformation mixture onto LB(Amp) plates prewarmed to 37° C.;
Incubate plates at 37° C. overnight;
Analyse the resulting transformands after minipreparation (3.2.2.1) with suitable enzymes in an analytical restriction digestion.

### EXAMPLE 4

Gene Expression in E. Coli Cells

The procedure is outlined as follows:
The plasmid is isolated from successfully sequenced clones and transformed into the expression strain W3110
A clone is picked from the transformation plate and used to prepare a 5 ml ON preculture
The preculture is streaked onto an LB(Amp) plate, and clones from this plate are used to inoculate expressions to be carried out later
1 ml of the preculture is then used to inoculate the 50 ml main culture (ratio 1:50) and the OD₆₀₀ is determined (reference measurement with uninoculated LB(Amp) medium)
The main culture (in a 200 ml Erlenmeyer flask) is incubated at 37° C. and 220 rpm
The OD₆₀₀ is determined every 30 min
Once the OD reaches 0.5, the cells are induced with 10 μl of anhydrotetracycline (1 mg/ml) per 50 ml of cell suspension (f.c. 0.2 μg of anhydrotetracycline per ml of cell suspension), and the OD is again determined (0 value)
The OD is determined every hour and growth is stopped 3 h after the time of induction
1 ml of thoroughly mixed bacterial suspension is placed in a tube and centrifuged at 6000 rpm for 5 min (less suspension may also be used if necessary)
The supernatant is aspirated off and the pellet is homogenized in 100 μl of 1×red sample buffer;
The homogenate is boiled for 5 min, cooled on ice and briefly centrifuged;
10 μl of sample are loaded into each well of an SDS gel and the electrophoresis (3.2.16) is carried out;
The gel is stained by Coomassie blue staining and/or by the method of Example 5.
   CELL disruption:

Cells from a 50 ml overnight culture are centrifuged at 3500 rpm and 4° C. for 15 min. The resulting supernatant is poured away and the cells are resuspended in 40 ml of 100 mM Tris/HCl (pH 8.5). The suspended cells are disrupted using a French press in a 1 inch cylinder under 18,000 psi. This entails the cells being forced through a narrow orifice (<1 mm) and subjected to a sudden fall in pressure. The cells burst due to the pressure difference on passing through the orifice. The structure of the cellular proteins is retained during this. To avoid proteolytic degradation of the required protein, a protease inhibitor should be added immediately after the cell disruption. For this purpose, 1 tablet of the EDTA-free Complete™ Protease-Inhibitor Cocktail (Roche) is added to each 40 ml of protein solution and dissolved at RT. The subsequent centrifugation at 6000 rpm for 20 minutes removes the cell detritus and large parts of DNA and RNA. The samples are then frozen at −20° C.

### EXAMPLE 5

Activity Staining of the GlcDH Band in the SDS Gel:

The glucose dehydrogenase band can be specifically detected in the SDS gel using iodophenylnitrophenyl-phenyltetrazolium chloride (INT). This is possible only because the SDS treatment does not destroy the GlcDH activity.

The GlcDH is detected by means of a colour reaction.

This entails the hydrogen formed in the reaction being transferred to the tetrazolium salt INT, producing a violet formazan. Phenanzine methosulfate serves as electron transfer agent.

Preincubation Buffer (0.1 M Tris/HCl, pH 7.5)

15.76 g of Tris/HCl
ad 1 l ddH₂O, pH 7.5 with NaOH

Reaction buffer (0.08% INT, 0.005% phenanzine methosulfate, 0.065% NAD, 5% Glc in 0.1 M Tris/HCl (pH 7.5)

| | | |
|---|---|---|
| 0.8 | g | of iodophenylnitrophenyltetrazolium chloride |
| | | (INT) |
| 0.05 | g | of methylphenazinium methosulfate |
| | | (phenanzine methosulfate) |
| 0.65 | g | of NAD |
| 50 | g | of D-(+)-glucose monohydrate (Glc) |
| ad 1 | l | 0.1 M Tris/HCl (pH 7.5) |

Storage Buffer for GlcDH:
26.5 g of EDTA
15 g of Na₂HPO₄
ad 1 l, pH 7.0 (NaOH)
   Sample Preparation:
   Dilute samples and markers in sample buffer.
   Boil in a water bath for 3 min and cool on ice, and centrifuge.
      SDS Gel Electrophoresis by Standard Methods.
      Activity Staining:
   Incubate SDS gel with fractionated protein bands in preincubation buffer at 37° C. with gentle shaking for 5 min
   Pour off buffer and cover with a sufficient amount of reaction buffer (RT), and incubate at 37° C. with gentle shaking (change buffer at least 1×)
   After incubation for about 30 min, the bands with GlcDH are stained red-violet.
   Wash gel in preincubation buffer, photograph and dry
   If required, carry out a subsequent Coomassie staining and then dry the gel.

### EXAMPLE 6

Immunological Detection Using the ECL System (Western Exposure™ Chemiluminescent Detection System):

Proteins coupled to a His tag are detected indirectly using two antibodies. The first Ab employed is the anti-^{RGS.}His antibody (QIAGEN) for detecting 6xHis-tagged proteins. The resulting antigen-antibody complex is then detected using the peroxidase (POD)-labelled AffiniPure goat anti-mouse IgG (H+L) antibody. After addition of the ECL substrate mixture, the bound peroxidase results in a chemiluminescent product which can be detected using a high performance chemiluminescence film.

Ponceau S Solution (0.5% Ponceau S, 7.5% TCA)

1.25 g of Ponceau S
18.75 g of TCA
Make up to 250 ml with double-distilled water.
   10×PBS Buffer pH 7.4
14.98 g of disodium hydrogen phosphate×2H₂O
2.13 g of potassium dihydrogen phosphate
87.66 g of sodium chloride
Make up to 1 l, check that pH is 7.4.
The 1× concentration of the buffer is employed.
   Biometra Blot Buffer
25 mM Tris
150 mM Glycine
10% Methanol
   Blocking Reagent
5% Skimmed milk powder
Dissolve in 1×PBS buffer.
   Washing Buffer
0.1% Nonidet™ P-40 (Sigma)
Dissolve in 1×PBS buffer

The detection was carried out as follows:
Cut a PVDF membrane (Immobilon P, Millipore) and 6×blotting filter paper to the size of the gel
Equilibrate the PVDF membrane for 15 sec in methanol and then in Biometra blot buffer, and apply the same procedure to the SDS gel and the filter papers
Blot construction: assemble 3 layers of filter paper, membrane, gel, 3 layers of filter paper in the blot chamber (air bubbles between the layers must be expelled otherwise no protein transfer takes place at these points)
Blotting: 1-1.5 mA/cm² of gel for 1 h

Check of Protein Transfer:
After the blotting, the protein transfer to the PVDF membrane is checked by staining with Ponceau S: incubate the membrane with 0.5% Ponceau S solution in a dish with gentle shaking for at least 2 min. Pour off dye (reusable) and destain the membrane under running deionized water. In this case, only strong protein bands are stained. The molecular weight marker is marked with a ballpoint pen.

Development of the Blot:

All incubations should be carried out in a dish on a Celloshaker and in a roller cabinet in 50 ml Falcon tubes since the membrane must never dry out during the following steps.
(1) Saturation
   30 min at 37° C. in a roller cabinet with PBS/5% skimmed milk powder
(2) 1^{st} antibody: incubate diluted 1:2000 in PBS/5% skimmed milk powder (volume about 7 ml/membrane) at 37° C. for 1 h
(3) Washing: Wash membrane copiously with washing solution PBS/0.1% NP-40 wash for 3×5 min
(4) POD-labelled Ab: incubate diluted 1:1000 in PBS/5% skimmed milk powder (new tube) at 37° C. for 1 h
(5) Washing: Wash membrane copiously with washing solution PBS/0.1% NP-40 wash for 3×5 min
(6) Development: Swirl membrane thoroughly (do not allow to dry) and place on a plastic sheet, cover completely with ECL developer solution (Amersham) for 1 min, swirl membrane and place in a doubled sheet, lay polaroid Hyperfilm on top and develop

### EXAMPLE 7

Tridegin Detection by Inhibition of Factor XIIIa (Method of Finney et al., 1997, Modified According to the Invention)

In place of the natural substrate of factor XIIIa, namely amino-containing side chains of amino acids, synthetic amines are also incorporated into suitable protein substrates. These synthetic amines have intramolecular markers which make detection possible. The amine incorporation test is a solid-phase test.

The titre plates are coated with casein. The substrate biotinamidopentylamine is incorporated into this casein by factor XIIIa. The casein-biotinamidopentylamine product can be detected by the streptavidin-alkaline phosphatase fusion protein (strep/AP). This sandwich can take place [sic] by detecting the phosphatase activity using p-nitrophenyl phosphate. This involves the following reaction:

4-Nitrophenyl phosphate + H₂O AP phosphate +
4-nitrophenolate [sic]

The formation of 4-nitrophenolate [sic] is determined by photometry at 405 nm and is directly proportional to the AP activity. The high-affinity interaction of biotin and streptavidin means that the phosphatase activity is likewise proportional to the factor XIIIa activity, that is to say a stronger absorption (yellow coloration) means a higher factor XIIIa activity (Janowski, 1997). EDTA is a very nonspecific inhibitor of factor XIIIa, whose cofactor Ca²⁺ is bound by EDTA in a chelate complex. For this reason, the protein samples used must not contain any EDTA and were pretreated with an EDTA-free protease inhibitor cocktail (Boehringer).

| | |
|---|---|
| Washing buffer: | 100 mM Tris/HCl, pH 8.5 |
| Solution A: | Dissolve 0.5% skimmed milk |
| | powder in washing buffer |
| Solution B: | Dissolve 0.5 mM biotin- |
| | amidopentylamine, 10 mM DTT, |
| | 5 mM CaCl₂ in washing buffer |
| Solution C: | Dissolve 200 mM EDTA in |
| | washing buffer |
| Solution D: | Dissolve 1.7 μg/ml of |
| | streptavidin-alkaline |
| | phosphatase in solution A |
| Solution E: | Dissolve 0.01% (w/v) Triton X- |
| | 100 in washing buffer |
| Solution F: | Dissolve 1 mg/ml p-nitrophenyl |
| | phosphate, 5 mM MgCl₂ in |
| | washing buffer |

Coating:
Distribute 200 μl of solution A in each well on a titre plate, depending on the number of samples
Shake at 37° C. for 30 min (Thermoshaker)
   Washing:
   Wash twice with 300 μl of washing buffer per well
      Incorporation Reaction:
   Distribute 10-150 μl of sample per well and add 5 μl of factor XIIIa per well and 200 μl of solution B per well
Shake at 37° C. for 30 min
   Stopping:
   Wash twice with 300 μl of solution C (factor XIIIa inhibition) per well
   Wash twice with 300 μl of washing buffer per well
      Strep/Ap Binding (Specific):
   Add 250 μl of solution D per well
   Incubate at RT for 60 min
      Washing:
   Wash with 300 μl of solution E per well (detaches the proteins which are not covalently bonded)
   Wash 4 times with 300 μl of washing buffer per well
      Substrate:
   Add 50 μl of solution F per well+200 μl of washing buffer per well
   Incubate at RT for 30 min
Measure with computer-assisted evaluation in a microtitre plate reader at 405 nm

### EXAMPLE 8

### Sensitivity of GlcDH Detection

The stated amount of purified GlcDH was put on an SDS gel. After the run, the SDS gel was incubated in preincubatiuon buffer at 37° C. for 5 minutes. The buffer was discarded and the gel was shaken in reaction buffer at 37° C. In a further step the gel was stained with Coomassie blue.

Reaction buffer for 1 litre:
0.1M Tris/HCL, pH 7.5
0.5M NaCl
0.2% Triton X-100-
0.8 g of iodophenylnitrophenyltetrazolium chloride
0.05 g of methylphenazinium methosulfate
0.65 g of NAD
50 g of D-(+)-glucose monohydrate
   Preincubation Buffer:
0.1M Tris/HCl, pH 7.5
0.5M NaCl

1-17. (canceled)

18. A method for detecting gene expression of polypeptide X comprising expressing a recombinant fusion protein comprising glucose dehydrogenase linked with polypeptide X and detecting the presence of glucose dehydrogenase, wherein the presence of glucose dehydrogenase correlates with gene expression of polypeptide X.

19. The method of claim 18, wherein glucose dehydrogenase is detected with an antibody that recognizes an epitope of glucose dehydrogenase.

20. The method of claim 18, wherein glucose dehydrogenase is detected by assaying for glucose dehydrogenase activity.

21. The method of claim 18, wherein glucose dehydrogenase is detected by i) fractionating the fusion protein by gel electrophoresis and ii) assaying for glucose dehydrogenase activity in the gel by incubating the gel in a solution containing a tetrazolium salt.

22. The method of claim 21, wherein said assay for glucose dehydrogenase activity produces a color.

23. The method of claim 22, wherein a tetrazolium salt is used to assay for glucose dehydrogenase activity.

24. The method of claim 23, wherein said tetrazolium salt is iodophenylnotrophenyl-phenyltetrazolium or nitro blue tetrazolium.

25. The method of claim 22, wherein said color reaction for the detection of glucose dehydrogenase activity is followed by protein staining.

26. The method of claim 25, wherein said protein stain is a silver stain or a coomassie blue stain.

27. A method for detecting a polypeptide X/polypeptide X interaction comprising expressing a recombinant fusion protein comprising glucose dehydrogenase linked with polypeptide X, contacting the expressed fusion protein with polypeptide X and detecting the polypeptide X/polypeptide X homodimer.

28. A method for detecting a polypeptide X/polypeptide Y interaction comprising expressing a recombinant fusion protein comprising glucose dehydrogenase linked with polypeptide X, contacting the expressed fusion protein with polypeptide Y and detecting the polypeptide X/polypeptide Y heterodimer.

29. A method for determining optimal protein expression conditions for polypeptide X comprising expressing, under specified expression conditions, a recombinant fusion protein comprising glucose dehydrogenase and polypeptide X and detecting glucose dehydrogenase.

30. The method of claim 29 wherein glucose dehydrogenase is detected with an antibody that recognizes an epitope of glucose dehydrogenase, wherein expression levels of glucose dehydrogenase correlate to expression levels of polypeptide X.

31. The method of claim 29 wherein glucose dehydrogenase is detected by assaying for glucose dehydrogenase activity, wherein levels of glucose dehydrogenase activity correlate to expression levels of polypeptide X.

32. The method of claim 31, wherein glucose dehydrogenase is detected by i) fractionating the fusion protein by gel electrophoresis and ii) assaying for glucose dehydrogenase activity in the gel by incubating the gel in a solution containing a tetrazolium salt.

33. The method of claim 32, wherein said assay for glucose dehydrogenase activity produces a color.

34. The method of claim 33, wherein a tetrazolium salt is used to assay for glucose dehydrogenase activity.

35. The method of claim 34, wherein said tetrazolium salt is iodophenylnotrophenyl-phenyltetrazolium or nitro blue tetrazolium.

36. The method of claim 33, wherein said color reaction for the detection of glucose dehydrogenase activity is followed by protein staining.

37. The method of claim 36, wherein said protein staining is silver staining or coomassie blue staining.

38. A method for increasing correct protein folding for protein X comprising expressing a recombinant fusion protein comprising glucose dehydrogenase and protein X, wherein correct protein X folding is higher in said fusion protein relative to protein X alone.

39. A method for increasing the solubility of protein X comprising expressing a recombinant fusion protein comprising glucose dehydrogenase and protein X, wherein the solubility of protein X is higher in said fusion protein relative to protein X alone.


SEQUENCE LISTING (17 sequences)

SEQ ID NO: 1 - DNA, 3992 bp, Bacillus megaterium
  CDS (186)..(968): Glucose Dehydrogenase from Bacillus megaterium
ccatcgaatggccagatgattaattcctaatttttgttgacactctatcattgatagagttattttaccactccctatcagtgatagagaaaagtgaaatgaatagttcgacaaaaatctagataacgagggcaatcgatgaattcgagctcggtacccggggatccctcgaggtcgacctgcagatgtatacagatttaaaagataaagtagttgtaattacaggtggatcaacaggtttaggacgcgcaatggctgttcgtttcggtcaagaagaagcaaaagttgttattaactattacaacaatgaagaagaagctctagatgcgaaaaaagaagtagaagaagcaggcggacaagcaatcatcgttcaaggcgatgtaacaaaagaagaagacgttgtaaatcttgttcaaacagctattaaagaatttggtacattagacgtaatgattaacaacgctggtgttgaaaacccagttccttctcatgagctatctctagataactggaacaaagttattgatacaaacttaacaggtgcattcttaggaagccgtgaagcaattaaatacttcgttgaaaacgacattaaaggaaatgttatcaacatgtctagcgttcacgaaatgattccttggccattatttgttcactacgcagcaagtaaaggcggtatgaaactaatgacggaaacattggctcttgaatatgcgccaaaaggtatccgcgtaaataatattggaccaggtgcgatgaacacaccaattaacgcagagaaatttgcagatccagaacaacgtgcagacgtagaaagcatgattccaatgggttacatcggtaaaccagaagaagtagcagcagttgcagcattcttagcttcatcacaagcaagctatgtaacaggtattacattatttgcagatggcggtatgacgaaatacccttctttccaagcaggaagaggctaatagagcgctatgagaggatcgcatcaccatcaccatcactaatagaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 2 - protein, 261 aa, Bacillus megaterium
MYTDLKDKVVVITGGSTGLGRAMAVRFGQEEAKVVINYYNNEEEALDAKKEVEEAGGQAIIVQGDVTKEEDVVNLVQTAIKEFGTLDVMINNAGVENPVPSHELSLDNWNKVIDTNLTGAFLGSREAIKYFVENDIKGNVINMSSVHEMIPWPLFVHYAASKGGMKLMTETLALEYAPKGIRVNNIGPGAMNTPINAEKFADPEQRADVESMIPMGYIGKPEEVAAVAAFLASSQASYVTGITLFADGGMTKYPSFQAGRG

SEQ ID NO: 3 - protein, 11 aa, Bacillus megaterium
AMRGSHHHHHH

SEQ ID NO: 4 - DNA, 4193 bp, Bacillus megaterium / Heamenteria ghilianii fusion
  CDS (141)..(344): Tridegin
ccatcgaatggccagatgattaattcctaatttttgttgacactctatcattgatagagttattttaccactccctatcagtgatagagaaaagtgaaatgaatagttcgacaaaaatctagataacgagggcaatcgatatgaaactattgccttgcaaagaatggcatcaaggtattcctaaccctaggtgctggtgtggggctgatctagaatgcgcacaagaccaatactgtgccttcatacctcaatgtagaccaagatcagaactgattaaacctatggatgatatataccaaagaccagtcgagtttccaaaccttccattaaaacctagggaggaaagcgctatgagaggatcgcatcaccatcaccatcacctgcagatgtatacagatttaaaagataaagtagttgtaattacaggtggatcaacaggtttaggacgcgcaatggctgttcgtttcggtcaagaagaagcaaaagttgttattaactattacaacaatgaagaagaagctctagatgcgaaaaaagaagtagaagaagcaggcggacaagcaatcatcgttcaaggcgatgtaacaaaagaagaagacgttgtaaatcttgttcaaacagctattaaagaatttggtacattagacgtaatgattaacaacgctggtgttgaaaacccagttccttctcatgagctatctctagataactggaacaaagttattgatacaaacttaacaggtgcattcttaggaagccgtgaagcaattaaatacttcgttgaaaacgacattaaaggaaatgttatcaacatgtctagcgttcacgaaatgattccttggccattatttgttcactacgcagcaagtaaaggcggtatgaaactaatgacggaaacattggctcttgaatatgcgccaaaaggtattcgcgtaaataatattggaccaggtgcgatgaacacaccaattaacgcagagaaatttgcagatccagaacaacgtgcagacgtagaaagcatgattccaatgggttacatcggtaaaccagaagaagtagcagcagttgcagcattcttagcttcatcacaagcaagctatgtaacaggtattacattatttgcagatggcggtatgacgaaatacccttctttccaagcaggaagaggctaatagagcgctatgagaggatcgcatcaccatcaccatcactaatagaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattgatagactggatggaggcggataaagttgcaggaccacttctgcgctcggcccttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaggaattaatgatgtctcgtttagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgcagtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcactagtttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgacccgaca

SEQ ID NO: 5 - protein, 68 aa, Bacillus megaterium / Heamenteria ghilianii fusion
MKLLPCKEWHQGIPNPRCWCGADLECAQDQYCAFIPQCRPRSELIKPMDDIYQRPVEFPNLPLKPREE

SEQ ID NO: 6 - protein, 261 aa, Bacillus megaterium / Heamenteria ghilianii fusion; identical to SEQ ID NO: 2

SEQ ID NO: 7 - protein, 11 aa, Bacillus megaterium / Heamenteria ghilianii fusion
AMRGSHHHHHH

SEQ ID NO: 8 - DNA, 32 bp, artificial sequence
  primer
gcgcgaattcatgtatacagatttaaaaagat

SEQ ID NO: 9 - DNA, 31 bp, artificial sequence
  primer
gcgcttcgaactattagcctcttcctgcttg

SEQ ID NO: 10 - DNA, 31 bp, artificial sequence
  primer
gcgcctgcagatgtatacagatttaaaagat

SEQ ID NO: 11 - DNA, 31 bp, artificial sequence
  primer
gcgcagcgctctattagcctcttcctgcttg

SEQ ID NO: 12 - DNA, 31 bp, artificial sequence
  primer
gcgcatcgatatgaaactattgccttgcaaa

SEQ ID NO: 13 - DNA, 31 bp, artificial sequence
  primer
gcgcctgcaggtgatggtgatggtgatgcga

SEQ ID NO: 14 - DNA, 22 bp, artificial sequence
  primer
ccatcgaatggccagatgatta

SEQ ID NO: 15 - DNA, 21 bp, artificial sequence
  primer
tagcggtaaacggcagacaaa

SEQ ID NO: 16 - DNA, 20 bp, artificial sequence
  primer
taatacgactcactataggg

SEQ ID NO: 17 - DNA, 18 bp, artificial sequence
  primer
tagaaggcacagtcgagg